(19) United States
(12) Patent Application Publication
(10) Pub. No.: US 2025/0281040 A1
(43) Pub. Date: Sep. 11, 2025

(54) DEVICE AND METHOD FOR ASSESSING VISUAL FUNCTION IN RELATION TO PUPILLARY REFLEX

(71) Applicant: M2S CO.,LTD, Seoul (KR)

(72) Inventors: Byung Jo KIM, Seoul (KR); Sun-Uk LEE, Seoul (KR); Jung Bin KIM, Seoul (KR); Young Wook YOON, Seoul (KR)

(73) Assignee: M2S CO.,LTD, Seoul (KR)

(21) Appl. No.: 18/270,123

(22) PCT Filed: Jul. 16, 2021
(86) PCT No.: PCT/KR2021/009154

§ 371 Date: Dec. 18, 2023

(30) Foreign Application Priority Data
Dec. 29, 2020 (KR) 10-2020-0186026

(51) Int. Cl. A61B 3/11; A61B 3/00; A61B 3/14

(52) U.S. Cl. CPC A61B 3/11; A61B 3/0025; A61B 3/14

(57) ABSTRACT

A device for assessing visual function in relation to pupillary reflex according to one embodiment includes an HMD worn on the head of a subject to be assessed to create a testing environment, and obtaining a pupil image of the subject to be assessed in the created testing environment; and an assessment module for calculating indicators for assessing visual function in relation to pupillary reflex by analyzing the pupil image of the subject to be assessed provided by the HMD, and assessing the visual function in relation to pupillary reflex on the basis of the calculated indicators.

### CROSS-REFERENCE TO PRIOR APPLICATIONS

This Application is a National Stage Patent Application of PCT International Application No. PCT/KR2021/009154 (filed on Jul. 16, 2021), which claims priority to Korean Patent Application No. 10-2020-0186026 (filed on Dec. 29, 2020), which are all hereby incorporated by reference in their entirety.

### BACKGROUND

The present application relates to a device and method for assessing visual function related to pupillary reflex.

### Background Art

A pupillary response test is intended to determine quantitative and qualitative changes in pupil size, and may be broadly divided into a static test and a dynamic test, through which a function of the optic nervous system may be assessed.

Here, the static test refers to observing the presence or absence of miosis and mydriasis and the presence or absence of anisocoria through light and dark room tests without using light.

Further, the dynamic test include a light reflex test to test pupillary reflex of an eye with or without direct light administration, an alternating light test to test for relative afferent pupillary defect, and a near reflex test to observe convergence, accommodation, and miosis that occur during near-focus.

However, these conventional test methods not only have limitations in creating test conditions, but also have limitations in that it is difficult to provide objective assessment results due to the absence of a method to quantify assessment results.

### SUMMARY

Therefore, in this technical field, there is a need for a method to provide more objective assessment standards by creating more accurate test conditions and quantifying assessment results in visual function assessment.

In order to solve the above problems, an embodiment of the present disclosure provides a device for assessing pupillary reflex-related visual function.

The device for assessing pupillary reflex-related visual function may include: an HMD configured to create a testing environment by being worn on a head of a subject of assessment, and obtain a pupil image of the subject of assessment in the created testing environment; and an assessment module configured to calculate an indicator for assessing pupillary reflex-related visual function by analyzing the pupil image of the subject of assessment provided from the HMD and assess pupillary reflex-related visual function based on the calculated indicator.

In addition, another embodiment of the present disclosure provides a method for assessing pupillary reflex-related visual function.

The method for assessing pupillary reflex-related visual function may include creating a testing environment through an HMD worn on a head of a subject of assessment; obtaining a pupil image of the subject of assessment in the testing environment created through the HMD; calculating pupil size of both eyes by analyzing the pupil image of the subject of assessment; calculating an indicator for assessing pupillary reflex-related visual function based on the calculated pupil size; and assessing pupillary reflex-related visual function of the subject of assessment based on the calculated indicator.

In addition, the above means for solving the problems do not enumerate all the features of the present disclosure. Various features of the present disclosure and their resulting advantages and effects may be understood in detail by referring to the specific embodiments below.

According to an embodiment of the present disclosure, by utilizing an HMD to assess the pupillary reflex-related visual function, it is possible to create more accurate test conditions and quantify assessment results to provide more objective assessment standards.

### BRIEF DESCRIPTION OF THE DRAWINGS

FIG. 1 is a configuration diagram of a device for assessing pupillary reflex-related visual function according to an embodiment of the present disclosure.

FIG. 2 is a flowchart of a method for assessing pupillary reflex-related visual function according to another embodiment of the present disclosure.

FIG. 3 is an example of assessing relative afferent pupillary defect according to an embodiment of the present.

FIG. 4 is an example of assessing pupillary light-near dissociation according to an embodiment of the present disclosure.

### EXPLANATION OF SYMBOLS

100: Pupillary reflex-related visual function assessment device
110: HMD
120: Assessment module
121: Pupil image analysis unit
122: Indicator calculation unit
123: Function assessment unit

### DETAILED DESCRIPTION

Hereinafter, with reference to the accompanied drawings, preferred embodiments will be described in detail so that those skilled in the art may easily practice the present disclosure. However, in describing preferred embodiments of the present disclosure in detail, if it is determined that a detailed description of a related known function or configuration may unnecessarily obscure the gist of the present disclosure, the detailed description will be omitted. In addition, the same symbols are used throughout the drawings for parts that perform similar functions and actions.

In addition, throughout the specification, when a part is said to be ‘connected’ to another part, this does not only include a case where they are ‘directly connected’, but also a case where they are ‘indirectly connected’ with another element in between. In addition, ‘comprising’ a component means that other components may be further included rather than excluding other components, unless specifically stated to the contrary.

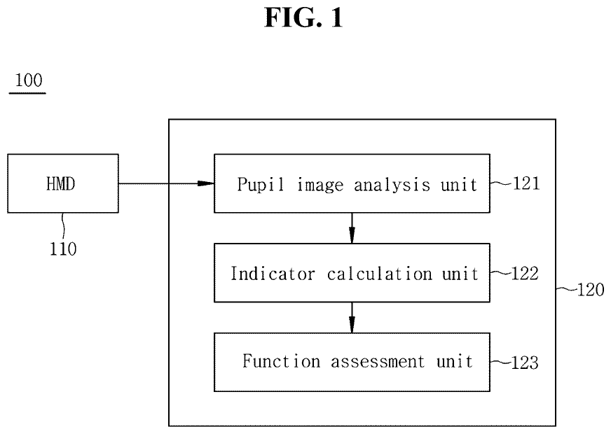

FIG. 1 is a configuration diagram of a device for assessing pupillary reflex-related visual function according to an embodiment of the present disclosure.

Referring to FIG. 1, a pupillary reflex-related visual function assessment device 100 according to an embodiment of the present disclosure may be configured to include a head mounted display (HMD) 110 and an assessment module 120.

The HMD 110 is worn on the head of a subject of assessment to create a testing environment for assessing pupillary reflex-related visual function, and to obtain a pupil image of the subject of assessment in the created testing environment.

According to an example, the HMD 110 may create the testing environment by alternately applying light stimulation to both eyes of the subject of assessment to assess relative afferent pupillary defect (RAPD). In this way, when light stimulation is applied by the HMD 110, the intensity of the light stimulation applied to both eyes, i.e., the illuminance, may be kept constant in multiple stages, and through this, more accurate test results may be expected.

According to another example, the HMD 110 may present a virtual object for the subject of assessment to gaze at in order to assess pupillary light-near dissociation (LND), and in this case, the testing environment may be created by maintaining the illuminance below a preset value to minimize light reflex due to light stimulation. Here, the preset value is the minimum illuminance value to minimize pupillary response due to light stimulation and may be appropriately set by an assessor. As an alternate method, in addition to the minimum illuminance value, normal values of light reflex for each illuminance may be obtained, and pupil miosis due to pure near reflex in the preset illuminance environment may also be quantitatively obtained. In this way, a fixation point may be presented while minimizing interference of the pupillary response due to the light stimulation through the HMD 110, and by bringing this fixation point closer to the subject of assessment, quantitative data on pupil miosis and near point of convergence (NPC) according to the near reflex of both eyes may be extracted, as described later. Here, by gradually changing the size of the fixation point presented through the HMD 110 to become larger, the fixation point may be made to appear to be approaching the subject of assessment, and in this case, the impact of the illuminance of the fixation point may be minimized by changing the illuminance of the fixation point itself as the fixation point gradually becomes larger (for example, lowering the illuminance in inverse proportion to the size of the fixation point).

Meanwhile, the HMD 110 may obtain the pupil image of the subject of assessment under the created testing environment and provide to the assessment module 120, which will be described later.

The assessment module 120 is to calculate indicators for assessing pupillary reflex-related visual function by analyzing the pupil image of the subject of assessment provided from the HMD 110 and assess pupillary reflex-related visual function based thereon, and may be configured to include a pupil image analysis unit 121, an indicator calculation unit 122, and a function assessment unit 123.

The pupil image analysis unit 121 may calculate the pupil size of both eyes by analyzing the pupil image of the subject of assessment and, if necessary, calculate the constriction velocity. Here, the method of analyzing the pupil image to calculate the pupil size and constriction velocity may employ various image analysis techniques known to those skilled in the art, so detailed description thereof will be omitted.

The indicator calculation unit 122 may calculate indicators for assessing pupillary reflex-related visual function based on the pupil size calculated by the pupil image analysis unit 121.

According to an example, the indicator calculation unit 122 may calculate the ratio of the illuminance of the light stimulation applied to the healthy side and the affected side, i.e., Light_{healthy_eye}/Light_{affected_eye}, when degrees of pupillary reflex in both eyes reach a similar point as the first indicator based on the pupil size of both eyes when step-by-step light stimulation is applied alternately to both eyes to assess relative afferent pupillary defect. Here, Light_{healthy_eye} represents the illuminance of the light stimulation applied to the healthy side, and Light_{affected_eye} represents the illuminance of the light stimulation applied to the affected side.

To explain the pupil response specifically, when one eye is illuminated by light, in addition to a direct response of the eye illuminated by the light, an indirect or consensual response occurs in the other eye which is not illuminated by the light, causing both eyes to contract simultaneously.

If light stimulation is alternately applied to both eyes at regular intervals (e.g., 2 to 5 seconds), it is possible to identify which eye has a dilated pupil due to a relatively slow or no pupillary response, and based on this, it is possible to distinguish between the affected side (i.e., the eye with a poor pupillary reflex) and the healthy side (i.e., the eye with a normal response). For example, when the same light stimulation (e.g., 10 lux) is applied to both eyes, if the pupillary response of the right eye is lower than that of the left eye, the right eye is the affected side (or affected eye).

In this case, the light stimulation (e.g., 90, 80, 70, . . . lux) applied to the left eye, which is the healthy side, may be gradually weakened, and the same light stimulation (e.g., 100 lux) as before may be applied to the right eye, which is the healthy side. At this time, in the case that the light stimulation applied to the healthy eye is 60 lux and the light stimulation applied to the affected eye is 100 lux, if the pupillary response of the healthy side and the affected side becomes similar, it may be said that the degrees of pupillary reflex have reached the similar point, and in this case, the first indicator may be calculated as 60/100=0.6. Here, a degree of pupillary response may compare pupil size or constriction velocity, and if the difference in the degree of pupillary response between both eyes is less than “average of physiological rhythmic movement (hippos)+n * standard deviation (where n is 1 or 2)”, it may be determined that the similar point has been reached.

Furthermore, in addition to calculating the relative afferent pupillary defect indicator by comparing the light reflex of both eyes, the indicator calculation unit 122 may further calculate quantitative data of the relative light reflex abnormality in a single eye as the first indicator. In other words, when light stimulation is applied to a single eye, the case where the pupil fails to maintain miosis and opens apart from physiological rhythmic movement is called pupillary escape. Here, if the degree of pupil opening is greater than the “average of physiological rhythmic movement (hippos)+n * standard deviation (where n is 1 or 2)”, it may be determined to correspond to the pupillary escape.

According to another example, the indicator calculation unit 122 may calculate Pupilₙₑₐᵣ/Pupil_{light} as a second indicator by quantifying the degree of near reflex compared to the degree of reflection for light stimulation of a preset illuminance to assess pupillary light-near dissociation. Here, Pupil_{light} represents the degree of pupil miosis in a light background with the preset illuminance compared to the pupil size in darkness, and Pupilₙₑₐᵣ represents the degree of pupil miosis when the virtual object is presented in the light background.

To explain the pupil response in detail, in addition to the light reflex, which is a response to light, the pupil constricts when looking at a nearby object. However, a neurological circuit responsible for the light pupillary reflex and a neurological circuit responsible for the near pupillary reflex are somewhat different in the brain stem, and in some neurological diseases, the pupil does not respond to light and is dilated, but when a proximal stimulus is applied, the near reflex operates normally and the pupil constricts, which is called light-near dissociation.

In the present embodiment, with the subject of assessment wearing the HMD 110, the degree of pupil miosis in a light background with a preset illuminance compared to the pupil size in the dark (Pupil_{light}) is first measured, and afterwards, a virtual object is presented in the same light background and the degree of pupil miosis is measured when approaching from a distance to a near distance from the subject of assessment (Pupilₙₑₐᵣ), and the ratio thereof may be calculated as a second indicator.

Furthermore, the indicator calculation unit 122 may further assess the convergence of both eyes that occurs as the virtual object presented through the HMD 110 approaches the subject of assessment, and may further calculate quantitative data of the near point of convergence (NPC) in this case as the second indicator.

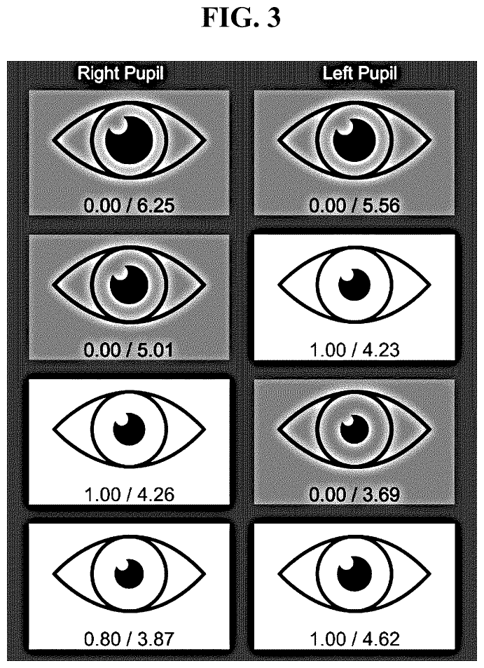

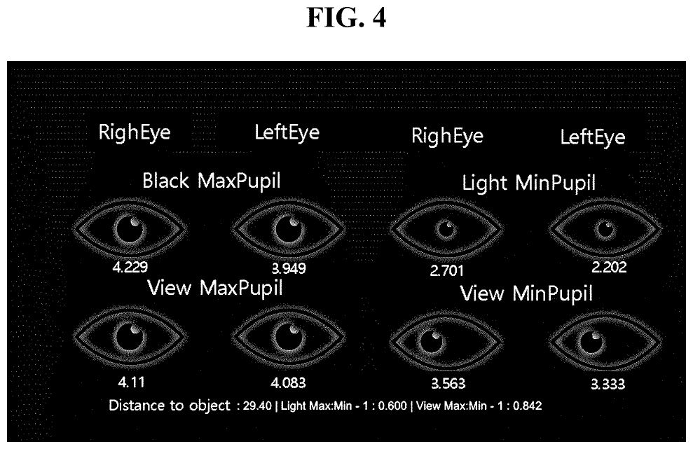

FIG. 3 is an example of assessing relative afferent pupillary defect according to an embodiment of the present disclosure, and FIG. 4 is an example of assessing pupillary light-near dissociation according to an embodiment of the present disclosure.

The function assessment unit 123 may assess the pupillary reflex-related visual function of the subject of assessment based on the indicators calculated by the indicator calculation unit 122.

For example, the function assessment unit 123 may quantitatively assess pupillary reflex-related visual function, particularly the function of the optic nerve (second cranial nerve), based on the first or second indicator calculated by the indicator calculation unit 122. For example, in the case of abnormality of the near reflex and light-near dissociation, the indicator calculated for a person with normal visual function may be used as a reference value to assess whether there is an abnormality. In addition, in the case of relative afferent pupillary defect, abnormalities in the optic nerves of one eye or both eyes may be assessed through indicators comparing both eyes and/or indicators in one eye.

The above-described assessment module 120 may be implemented by a processing device (e.g., server, mobile terminal, etc.) connected to the HMD 110 through wired or wireless communication.

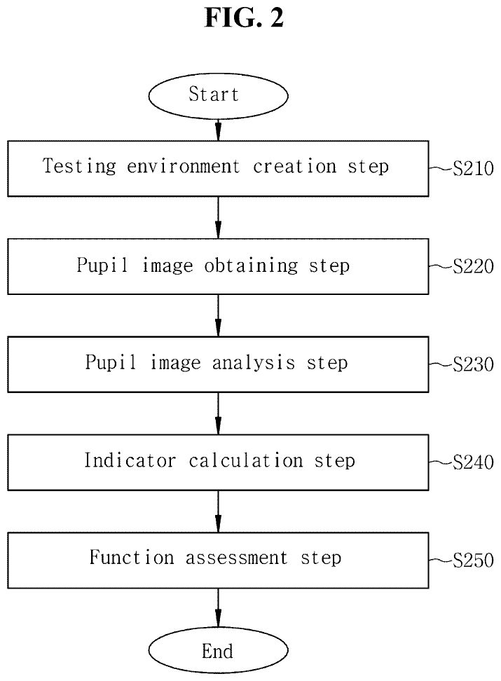

FIG. 2 is a flowchart of a method for assessing pupillary reflex-related visual function according to another embodiment of the present disclosure.

Referring to FIG. 2, the method for assessing pupillary reflex-related visual function according to another embodiment of the present disclosure may be configured including a testing environment creation step (S210), a pupil image obtaining step (S220), a pupil image analysis step (S230), an indicator calculation step (S240), and a function assessment step (S250).

Specifically, in the testing environment creation step (S210), a testing environment may be created through the HMD worn on the head of the subject of assessment.

In the pupil image obtaining step (S220), a pupil image of the subject of assessment in the testing environment created through the HMD may be obtained.

In the pupil image analysis step (S230), pupil size of both eyes may be calculated by analyzing the pupil image of the subject of assessment.

In the indicator calculation step (S240), indicators for assessing pupillary reflex-related visual function may be calculated based on the calculated pupil size.

In the function assessment step (S250), the pupillary reflex-related visual function of the subject of assessment may be assessed based on the calculated indicators.

The method for assessing pupillary reflex-related visual function described above with reference to FIG. 2 may be performed by the device for assessing pupillary reflex-related visual function shown in FIG. 1, and since specific details of each step are the same as described above with reference to FIG. 1, redundant description thereof will be omitted.

As a result of testing 10 patients with optic neuropathy and 20 normal people according to the embodiment of the present disclosure as described above, the results of assessing pupillary reflex-related visual function using the embodiment of the present disclosure showed a sensitivity of 100% and a specificity of 90% and it was confirmed that the results have excellent diagnostic value compared to the conventional test, which showed a sensitivity of 70% and a specificity of 90%.

The present disclosure is not limited to the above-described embodiments and accompanied drawings. It would be obvious to those skilled in the art to which the present disclosure pertains that the components according to the present disclosure may be replaced, modified, and changed without departing from the technical spirit of the present disclosure.

1. A device for assessing pupillary reflex-related visual function, the device comprising:
an HMD configured to create a testing environment by being worn on a head of a subject of assessment, and obtain a pupil image of the subject of assessment in the created testing environment; and
an assessment module configured to calculate an indicator for assessing pupillary reflex-related visual function by analyzing the pupil image of the subject of assessment provided from the HMD and assess pupillary reflex-related visual function based on the calculated indicator.

2. The device of claim 1, wherein the assessment module comprises:
a pupil image analysis unit configured to calculate pupil size of both eyes by analyzing the pupil image of the subject of assessment;
an indicator calculation unit configured to calculate the indicator for assessing pupillary reflex-related visual function based on the pupil size calculated by the pupil image analysis unit; and
a function assessment unit configured to assess the pupillary reflex-related visual function of the subject of assessment based on the indicator calculated by the indicator calculation unit.

3. The device of claim 2, wherein the HMD is configured to create the testing environment by alternately applying light stimulation to both eyes of the subject of assessment to assess relative afferent pupillary defect.

4. The device of claim 3, whereinthe indicator calculation unit is configured to calculate Light_{healthy_eye}/Light_{affected_eye}, which is a ratio of illuminance of the light stimulation applied to a healthy side to illuminance of the light stimulation applied to an affected side when degrees of pupillary reflex in both eyes reach a similar point, as a first indicator, based on the pupil size of both eyes when step-by-step light stimulation is applied alternately to both eyes to assess the relative afferent pupillary defect, and
wherein Light_{healthy_eye} represents the illuminance of the light stimulation applied to the healthy side, and Light_{affected_eye} represents the illuminance of the light stimulation applied to the affected side.

5. The device of claim 4, wherein the indicator calculation unit is configured to further calculate pupillary escape to quantitatively calculate a relative light reflex abnormality in a single eye as the first indicator.

6. The device of claim 2, wherein the HMD is configured to present a virtual object for the subject of assessment to gaze at in order to assess pupillary light-near dissociation, and create the testing environment by maintaining illuminance at a preset value.

7. The device of claim 6, wherein the indicator calculation unit is configured to calculate Pupilₙₑₐᵣ/Pupil_{light} as a second indicator by quantifying a degree of near reflex compared to a degree of reflection for light stimulation of a preset illuminance to assess pupillary light-near dissociation, and
wherein Pupil_{light} represents a degree of pupil miosis in a light background with the preset illuminance compared to pupil size in darkness, and Pupilₙₑₐᵣ represents a degree of pupil miosis when the virtual object is presented in the light background.

8. The device of claim 7, wherein the indicator calculation unit is configured to further calculate a near point of convergence as the second indicator.

9. A method for assessing pupillary reflex-related visual function, the method comprising:
creating a testing environment through an HMD worn on a head of a subject of assessment;
obtaining a pupil image of the subject of assessment in the testing environment created through the HMD;
calculating pupil size of both eyes by analyzing the pupil image of the subject of assessment;
calculating an indicator for assessing pupillary reflex-related visual function based on the calculated pupil size; and
assessing pupillary reflex-related visual function of the subject of assessment based on the calculated indicator.
